(19) United States
(12) Patent Application Publication
(10) Pub. No.: US 2023/0330013 A1
(43) Pub. Date: Oct. 19, 2023

(54) HYDROGEL COMPRISING GLYCEROL AND A CARBOMER FOR TREATING THE RESPIRATORY SYMPTOMS OF COVID-19 DISEASE VIA THE NASAL ROUTE

(71) Applicant: COSMOSOFT, Paris (FR)

(72) Inventors: Stéphanie AUDRAN, MERE (FR); Daniel GREFF, MERE (FR)

(21) Appl. No.: 18/040,468

(22) PCT Filed: Aug. 3, 2021
(86) PCT No.: PCT/EP2021/071696

§ 371 Date: Feb. 3, 2023

(30) Foreign Application Priority Data
Aug. 4, 2020 (FR) FR2008276
Oct. 27, 2020 (FR) FR2010995

(51) Int. Cl. A61K 9/06; A61K 47/10; A61K 47/32; A61K 9/00; A61P 31/14

(52) U.S. Cl. CPC A61K 9/06; A61K 47/10; A61K 47/32; A61K 9/0043; A61P 31/14

(57) ABSTRACT

The invention relates to a product for treating viral diseases, such as COVID-19, via the nasal route. The product comprises a hydrogel based on water, glycerol and a carbomer.

### TECHNICAL FIELD

The present invention relates to a product for treating viral diseases, in particular COVID-19, via the nasal route. This product comprises a hydrogel based on water, glycerol and a carbomer.

### PRIOR ART

The treatment of respiratory viral infections by nasal spraying of a liquid composition comprising a mixture of kappa-carrageenan, sodium chloride and sorbitol has been described in application WO 2017/009351. However, the sodium chloride concentrations in the products offered are very high, and cause irritation of the nasal mucosa which is very detrimental when the mucosa is weakened by an infection. In addition, under the force of the jet, the spraying of the composition propels the viruses present in the nasal cavities towards the pulmonary tracts, and risks aggravating the disease to be treated.

There remains a need for a more effective nasal viral treatment.

### GENERAL DESCRIPTION OF THE INVENTION

The invention meets this need by proposing a product comprising a hydrogel essentially consisting of water, glycerol and a carbomer.

In the present description, the term “essentially devoid” is understood to mean an amount of less than 0.1% by mass, preferably less than 0.05% by mass and even more preferably less than 0.01% by mass, the percentages being expressed either with respect to the mass of the hydrogel, or with respect to the mass of the product, as the case may be.

It has been surprisingly discovered that the product according to the invention allows to treat and prevent viral diseases.

The product of the invention advantageously allows to limit the infection by creating a film which prevents the virus from binding itself to the cells of the nasal mucosa and which also prevents the cycle of infection and replication from being initiated. The product prevents the virus from entering the cells of the nasal epithelium and infecting them. It also allows to promote hydration of the inflamed nasal mucosa, without disturbing the elimination of mucus.

The product thus allows to limit the entry of the coronavirus through the nose, and to limit its dissemination in the respiratory tract, in particular in the trachea and the lungs, during inspiration. It prevents aspiration of contaminating secretions from the upper respiratory tract into the lungs.

The epithelium of the nasal passages is protected from dehydration and inhaled viruses by the secretion of mucus, which constitutes a physical barrier and a filter to prevent foreign bodies from reaching the pulmonary alveoli. The product of the invention allows to protect the ciliated cells from infection, without blocking the secretion of mucus by the goblet cells. Finally, it does not increase the consistency of the mucus and does not disturb its evacuation.

The treatment of the disease is effective from the first days of the infection and it is easy to be applied.

Many viral diseases, including COVID-19 disease, primarily manifest as lung involvement of the bronchi, bronchioles, and alveoli. COVID-19 disease can progress to acute respiratory distress syndrome. The product of the invention acts upstream of the infection of the pulmonary cells by limiting the entry of the virus into the organism at the nose, more precisely by preventing its binding and its replication in the epithelial cells of the nasal cavities.

The product of the invention allows to completely eliminate the presence of viruses. It is therefore much more effective than the products of the prior art called “antiviral products” which reduce the viral load without totally eliminating the virus. The products of the prior art used to treat viral diseases via the nasal route have an anti-viral activity allowing to reduce the number of viruses, without however eliminating them completely.

### DETAILED DESCRIPTION OF THE INVENTION

The object of the invention is therefore a product for use thereof in the treatment or prevention of the respiratory symptoms of COVID-19 disease in humans, said product comprising a hydrogel based on water, glycerin and a carbomer, and being intended for application to the respiratory epithelium. The product is particularly adapted for topical application to the nasal mucosa.

Also described is a product for use thereof in the treatment of COVID-19 disease in humans caused by the SARS-CoV-2 coronavirus or a disease caused by one of the variants of this coronavirus. The product comprises a hydrogel which can be obtained by suspending a carbomer in a mixture essentially consisting, or even consisting, of water, glycerin and a carbomer. The product is intended for application to the respiratory epithelium.

It has in fact been surprisingly discovered that the product of the invention is much more effective than the products administered nasally proposed in the prior art for treating viral diseases. It has been discovered that the product of the invention blocks the replication of the SARS-CoV-2 virus by inhibiting certain enzymes involved in the process of penetration of the virus into the cells of the mucosa. In particular, the product of the invention inhibits the enzymatic activities of furin and of the angiotensin-converting enzyme 2 (ACE2) of the SARS-CoV-2 virus or its variants.

Furin is a serine protease which belongs to the family of proprotein convertases like subtilisin. This enzyme catalyzes the proteolytic maturation of proprotein substrates in the secretory pathway, and allows the inhibition of the replication of viruses that use furin to activate their glycoproteins, in particular coronaviruses and influenza viruses. The inventors have demonstrated that the product of the invention can inhibit more than 99% of the enzymatic activity of furin. By inhibiting the activity of furin, the product of the invention prevents the binding of the virus to the membrane proteins of host cells, and limits their infection.

Coronaviruses are characterized by a crown of proteins called “Spike” proteins or S proteins, and must undergo an activation step to infect the organism. This activation step, induced by an enzyme called furin, consists of cutting the Spike protein in order to make it functional. The Spike protein, once activated, attaches to one of the receptors present on the surface of the host human cell, this receptor being the angiotensin-converting enzyme 2 or ACE2.

Coronavirus disease 2019 (COVID-19) is caused by severe acute respiratory syndrome coronavirus 2 (SARS-CoV-2), whose mechanism of entry into the host cell involves activation by furin. The activated Spike viral protein can then be bound to ACE2 receptors found on the surface of type I and II pneumocytes, but also on endothelial cells and ciliated bronchial epithelial cells. Products targeting the interaction between the Spike protein of SARS-CoV-2 and ACE2 are therefore effective in fighting against COVID-19.

Inhibiting furin also helps control infections with avian influenza A viruses, which enter cells by cleavage of the glycoprotein hemagglutinin (HA). Low pathogenic avian influenza viruses contain a single basic amino acid at the cleavage site of the HA protein. The insertion of a furin cleavage site in the HA protein of the H5N1 avian influenza virus results in replication in multiple tissues and higher pathogenicity, due to the presence of furins in multiple tissues.

Angiotensin-converting enzyme 2 (ACE2) is an exopeptidase that catalyzes the conversion of angiotensin II to angiotensin 1-7 and L-phenylalanine. ACE2 was found to be one of the receptors for human respiratory coronavirus NL63, but also for SARS coronaviruses (SARS-CoV and SARS-CoV-2). The inventors have demonstrated that the product of the invention allows direct inhibition of the activity of the ACE2 protein up to 84%.

In conclusion, the product of the invention, which inhibits both the activity of furin and the activity of the ACE2 protein, proteins essential for intracellular viral translocation, therefore allows to fight against the diseases caused by viruses activated by furin and viruses that bind to the ACE2 protein of host cells.

The invention therefore relates to a product for use thereof in the treatment or prevention of respiratory symptoms of COVID-19 disease in humans, said product comprising a hydrogel based on water, glycerol and a carbomer, and said product being in a form adapted for topical application to the respiratory epithelium.

Hydrogel means within the meaning of the invention, a gel containing water and glycerin whose matrix is a network of carbomer. The hydrogel may be able to swell substantially in the presence of a large amount of water or an aqueous solution such as a biological fluid, in particular nasal mucus.

The hydrogel is capable of being obtained by suspending at least one carbomer in a mixture essentially consisting or consisting of water, glycerol. The term “essentially consisting” means a mixture in which the sum of the percentage by mass of water and the sum of the percentage of glycerol is greater than 99.0%, preferably greater than 99.5% and more preferably greater than 99.9% by mass relative to the mass of the mixture. After suspending the carbomer in the mixture essentially consisting or consisting of water and glycerol, a pH adjusting agent is preferably added. It is also possible to add to the suspension of the carbomer at least one polyol other than glycerin. The polyol may be selected from polyethylene glycols and 1,2-alkane-diols.

The polyethylene glycol is preferably non-hydroxylated.

The mass ratio between glycerol and water in the hydrogel or the mass ratio between glycerol and water in the carbomer suspension is preferably comprised between 0.8 and 10.0, for example between 1.0 and 5.0, between 1.0 and 3.0, between 1.5 and 3.5, or between 2.0 and 2.5. The hydrogel is preferably essentially devoid of any fatty substance and of any biologically active compound such as a zinc salt, alpha-pinene, methyl n-nonanone or a plant extract. In a particular embodiment of the invention, the hydrogel consists essentially of or consists of water, glycerin, at least one carbomer, at least one polyol different from glycerin and at least one pH adjusting agent.

The product of the invention may consist of the hydrogel or may consist of the hydrogel and at least one other ingredient selected from water, pH adjusters, and gelling agents other than the carbomer contained in the hydrogel. The hydrogel of the invention can therefore be mixed with water to prepare the product of the invention. The water which is present in the product can therefore come from the water contained in the hydrogel and from the water which has been optionally added to the hydrogel, once the latter has been prepared. The water present in the product can preferably represent from 70% to 98% by mass, preferably from 85% to 95% by mass of the mass of the product. In one embodiment of the invention, the hydrogel represents for example from 5% to 100%, from 5% to 90%, from 10% to 85%, from 10% to 70%, from 10% to 50%, from 10% to 40%, or preferably from 10% to 30% by mass of the mass of the product.

The carbomer is a compound with the INCI CARBOMER designation. It may have the following CAS numbers: 9007-20-9, 9003-01-4, 76050-42-5, 9062-04-8, 9007-16-3 or 9007-17-4.

The carbomer preferably comprises from 55% to 70%, more preferably from 56% to 68%, of carboxylic acid groups (—COOH). This percentage may be determined by any method known to the person skilled in the art, in particular an acid-base titration method in accordance with one of the methods described in the monographs relating to the carbomers of the American, European and Chinese pharmacopoeias.

According to one of these methods, a carbomer dispersion is titrated by potentiometry with sodium hydroxide using a glass-calomel electrode. The carboxylic acid content is calculated based on the volume of standard sodium hydroxide solution used.

In one embodiment, the carbomer is a homopolymer of acrylic acid cross-linked with an allyl ether of pentaerythritol, or a homopolymer of acrylic acid cross-linked with an allyl ether of sucrose.

The viscosity of the carbomer, measured at 0.5% by mass in water, at pH 7.5 and 25° C. (Brookfield RVT, 20 revolutions/min), preferably ranges from 35 000 cps to 70 000 cps, and more preferably from 40 000 cps to 60 000 cps. The viscosity of the carbomer can be measured by any method known to the person skilled in the art, in particular any method described in one of the monographs relating to the carbomers of the American, European and Chinese pharmacopoeias.

In a particular case, the carbomer is a homopolymer of acrylic acid cross-linked with an allyl ether of pentaerythritol, the name of which, according to the American pharmacopoeia, is Carbomer Homopolymer Type C. Such a carbomer is commercially available under the trademark CARBOPOL® 980 from Lubrizol.

In the hydrogel, the mass ratio between the carbomer and the glycerin is preferably comprised between 1/100 and 20/100, for example between 1/100 and 5/100 or 2/100 and 5/100.

The glycerol preferably represents from 30% by mass to 80% by mass of the hydrogel, and more preferably from 50% by mass to 75% by mass of the hydrogel, for example from 60% to 75% by mass of the hydrogel.

According to a particular embodiment of the invention, the hydrogel contains from 30% to 80% by mass of glycerol and the mass ratio between glycerol and water is comprised between 1.0 and 4.0. The hydrogel may contain at least one non-hydroxylated polyol different from glycerol, preferably selected from the group consisting of polyethylene glycols, preferably non-hydroxylated polyethylene glycols, and 1,2-alkane-diols. 1,2-octanediol can be used as 1,2-alkane-diol.

It is preferred to use non-hydroxylated polyols, since it has been discovered that hydroxylated polyols can be very irritating to the nasal mucosa. The product of the invention preferably contains less than 1% by mass, more preferably less than 0.1% by mass of a hydroxylated polyol. The product of the invention advantageously lacks said hydroxylated polyol.

The polyethylene glycols used in the product of the invention preferably have a mass-average molecular mass ranging from 200 g/mol to 1000 g/mol, or a number-average molecular mass ranging from 200 g/mol to 1000 g/mol. It is also preferred that the mass ratio between the glycerol and the non-hydroxylated polyol(s), in particular the mass ratio between the glycerol and the polyethylene glycol, be comprised between 1/1 and 3/1.

The hydrogel preferably contains from 5% to 50% by mass of water, for example from 10% to 40% or from 15% to 35%, and preferably from 20% to 30% by mass of water.

According to one embodiment, the hydrogel consists of water, glycerol, at least one carbomer (in particular the homopolymer of acrylic acid cross-linked with an allyl ether of pentaerythritol), at least one non-hydroxylated polyol and a pH adjuster. The hydrogel can be made by suspending the carbomer, in particular the homopolymer of acrylic acid cross-linked with an allyl ether of pentaerythritol, in a mixture comprising water and glycerol, in particular a mixture consisting of water and of glycerol.

In a particular example, the product of the invention comprises more than 99% by mass of a mixture consisting of water, at least one non-hydroxylated polyol, glycerol, a carbomer, such as a homopolymer of acrylic acid cross-linked with an allyl ether of pentaerythritol, and a pH adjuster.

Advantageously, the product of the invention does not contain steroidal or non-steroidal anti-inflammatory compounds generally used in the prior art. It has in fact been surprisingly discovered that the product of the invention has an anti-inflammatory activity on the nasal mucosa, in the absence of any active principle penetrating into the metabolism.

The pH of the hydrogel and/or the pH of the product of the invention preferably ranges from 5.5 to 6.5, and can be of the order of 6.0.

According to a particular embodiment, the hydrogel comprises a mucopolysaccharide. Mucopolysaccharides, in accordance with the definition given in the dictionary of the Academy of Medicine, also bear the name of glycosaminoglycans. They are formed of repeating units derived from a monosaccharide (galactose or a uronic acid such as iduronic acid or glucuronic acid) and a disaccharide comprising a sulfated or non-sulfated hexosamine.

Among the mucopolysaccharides which can be used in the context of the invention are hyaluronic acid, chondroitin-sulfates, heparan-sulfates, heparin, dermatan-sulfates and keratan-sulfates. Sulfated mucopolysaccharides can be bound to proteins through a short glycan sequence to form a proteoglycan.

The mucopolysaccharide is advantageously provided in the product by an ingredient of natural origin containing the same. The product of the invention may contain from 1% to 10% by mass of mucopolysaccharide(s), for example of the order of 5% by mass.

The product of the invention advantageously contains very small amounts of preservative(s), such as for example ethylene-diamine-tetraacetic, phenoxyethanol, potassium sorbate and parabens such as ethyl parabens. It is preferably essentially devoid thereof, or even completely devoid thereof. In particular, the hydrogel and the product of the invention are essentially devoid of, or even devoid of, ethanol, which is excessively irritating for the mucous membranes.

Unlike most of the products used in the prior art to treat diseases affecting the nasal mucosa, the product of the invention contains amounts of sodium chloride of less than 0.1% by mass. It is advantageously devoid thereof, because sodium chloride is irritating and drying for the nasal mucous membranes. Its irritating power is all the more harmful when the mucous membrane is inflamed.

In one embodiment of the invention, the product of the invention is essentially devoid of any biologically active compound. It is advantageously devoid of ions or molecules having an anti-viral activity and/or an anti-inflammatory activity, such as for example zinc salts, plant extracts, alpha-pinene or methyl n-nonanone. It has indeed been discovered very surprisingly and contrary to the teaching of the prior art suggesting the necessary presence of an anti-viral or anti-inflammatory pharmaceutical active ingredient, that a hydrogel comprising water, glycerol and a carbomer devoid of a biologically active compound can treat diseases caused by SARS-CoV2 and its variants.

The product may be in any form adapted for nasal application, preferably in a form adapted for topical application to the epithelium of the nasal mucosa, in the nasal cavities, or to the epithelium of the oropharynx (back of the nasal cavities). The product can thus be applied using a local application means such as a disposable cotton swab, a cannula or a mild nasal spray. It is preferred to use a disposable cotton swab within the scope of the invention.

The viscosity of the product is preferably comprised between 40 000 cps and 100 000 cps, for example between 50 000 cps and 90 000 cps, when measured at 25° C. with a Brookfield DV+PRO device equipped with an RV06 needle, at a speed of 3 revolutions per minute, the measurement being carried out 1 minute after the start of rotation.

The product is advantageously applied at the first symptoms of the disease to attenuate or suppress the viral load and treat the disease.

The product can be applied in the absence of any symptoms to prevent viral infection, especially to prevent coronavirus infection such as COVID-19.

The invention also relates to a method for manufacturing the product described above, said method comprising a step of preparing the hydrogel by suspending the carbomer in a mixture essentially consisting or consisting of water and glycerol.

A method for manufacturing a product according to the invention may comprise, in addition to the step of preparing the hydrogel as a first step, a second step of mixing the hydrogel obtained with at least one other ingredient selected from water, hydrophilic gelling agents known to the person skilled in the art, and biologically active products. The hydrogel obtained can therefore be mixed with water to prepare the product of the invention. A hydrophilic gelling agent is preferably different from the carbomer which was used to manufacture the hydrogel.

As part of the step of manufacturing the hydrogel, a mixture essentially consisting or consisting of water and glycerol is prepared, the mass ratio between the glycerol and the water preferably being comprised between 1/1 and 3/1, then the carbomer is dispersed in this mixture, preferably with stirring. The mass ratio between the glycerol and the water in the mixture used to prepare the hydrogel can be comprised between 1.0 and 4.0, preferably comprised between 1.0 and 3.0, more preferably comprised between 1.5 and 2.5. In the hydrogel manufacturing method, the mass ratio between the carbomer and the glycerol can be comprised between 1/100 and 10/100, preferably between 1/100 and 5/100, more preferably between 1/60 and 1/30.

After dispersing the carbomer in the mixture essentially consisting of water and glycerol, a pH adjusting agent is added, preferably in an amount sufficient to obtain a pH comprised between 5.5 and 6.5. After dispersing the carbomer in the mixture essentially consisting of water and glycerol, it is possible to add at least one polyol other than glycerol as described previously.

The product of the invention can be manufactured in at least two steps, including a first step of manufacturing the hydrogel comprising water, glycerol and carbomer, and a second step of mixing the hydrogel obtained with the other ingredient(s) which is/are possibly part of the composition of the product.

The second step may comprise the addition of at least one ingredient selected from the group consisting of water, a gelling agent, a mucopolysaccharide, a preservative, a pH adjusting agent. According to a particular embodiment, water, a gelling agent and a pH adjusting agent are added to prepare the product of the invention.

The product of the invention can be applied to a textile mask to provide it with a barrier function to the penetration of the virus into the body, or to improve its barrier function to the penetration of the virus into the body. The textile mask on which the product of the invention is applied may be known from the prior art. Also, the invention further relates to a protective mask made of textile material comprising the product as described above.

The mask of the invention may be made from textile materials selected from fabrics, knits, fibers, threads, braids and nonwovens,

In a particular embodiment, the mask is a KN 95 protective mask, an FFP2 mask or an FFP3 mask. FFP masks (abbreviation of the term “Filtering Face Piece Particles”) comprise three categories according to their filtration capacity, which is determined and validated by the European standard N149. An FFP2 mask filters at least 92% of airborne particles larger than 0.6 microns (that is to say it allows inward leakage between the mask and the face, with a maximum average of 8%), while an FFP3 mask filters at least 98% of airborne particles (that is to say it allows inward leakage between the mask and the face, with a maximum average of 2%).

An N95 FFP2 mask complies with the American standard N95, compliance with which is necessary to be able to be sold on American territory, and a KF94 FFP2 mask will comply with the standard to be respected for marketing in South Korea or China. The duration of use of the FFP2 mask varies from 3 to 8 hours.

In another embodiment, the protective mask of the invention may be a surgical mask, for example a surgical mask comprising at least three thicknesses, such as for example a Type II 3-ply mask in accordance with standard EN14683:2019, and optionally also in accordance with EN 14638:2016. The bacterial filtration of these masks can be greater than 98%, the degree of breathability (Delta P) can be greater than 3 Pa/cm².

Finally, the protective mask of the invention can also be a fabric mask, such as for example a fabric mask in accordance with UNS1 certification, in accordance with AFNOR S76-001 standard and certified washable 50 times by the independent organization IFTH. The fabric mask may have a filtration capacity equal to 100%.

The product of the invention can also be used to fight against infections caused by any type of virus attaching to the nasal mucosa, in particular coronaviruses other than SARS-CoV-2 such as SARS-CoV, the NL63 coronavirus, rhinoviruses, metapneumoviruses, syncytial viruses, seasonal influenza virus, avian influenza A virus, avian influenza H5N1 virus and adenoviruses.

The invention is illustrated by the following example.

### EXAMPLE 1: PREPARATION OF THE PRODUCT OF THE INVENTION

The product according to the invention is obtained from the following ingredients, the percentages being by mass. In accordance with the method of the invention, the hydrogel is prepared before optionally adding other ingredients. In this example, the ingredient added to the hydrogel is water.

Ingredients used to prepare the product: 10% of glycerol, 0.3% of a carbomer sold under the commercial reference CARBOPOL® 980 by the company Lubrizol and called Carbomer Homopolymer Type C according to the American Pharmacopoeia, 2% of PEG-400 (polyethylene glycol having a number-average molecular mass equal to 400 g/mol), 0.1% of NaOH 10 N, and the balance to 100% of purified water, the percentages being expressed by mass relative to the total mass of the product.

In a first step, the carbomer is dispersed in a mixture consisting of glycerin and a portion of purified water, the mass ratio between glycerin and water being equal to 1.4, with stirring at room temperature, then PEG-400 and NaOH are added. In a second step, the rest of the purified water is added to the hydrogel obtained. The product can optionally be filtered before packaging.

The product is applied to the nasal epithelium using a disposable cotton swab or nasal spray, in several people with the first symptoms of COVID-19, three to four times a day.

The product eliminates all the coronaviruses responsible for COVID-19 present in the nasal cavities.

### EXAMPLE 2: INHIBITION OF THE ACTIVITY OF FURIN CONVERTASE BY THE PRODUCT OF THE INVENTION

The purpose of this study is to evaluate the modulation of the activity of furin by the product of the invention in an acellular In Vitro model using the analysis kit sold under the brand name The Sensolyte® Rh110 Furin Assay Kit.

### Material

The product of the invention in accordance with Example 1 was tested in triplicate at three concentrations: 5%, 2% and 0.5%.

The kit sold under the brand name SensoLyte® Rh110 Furin Assay Kit allows continuous measurement of furin activity using a fluorogenic substrate. Upon cleavage by furin, this substrate generates bright green fluorescence. The reagents used are shown in Table 1 below.

TABLE 1
Used reagents

| REAGENT | SUPPLIER | REFERENCE |
|---|---|---|
| Sensolyte Rh110 Furin Activity Assay | ANASPEC | AS-72256 |
| Kit (Fluorimetric) | | |
| Component A = Rh110 furin substrate | ANASPEC | AS-72256 |
| Component C = Recombinant human furin | ANASPEC | AS-72256 |
| Component D = 2X Assay Buffer | ANASPEC | AS-72256 |
| Component E = Inhibitor | ANASPEC | AS-72256 |
| Water plus | Carlo Erba | 412142 |
| DMSO | Sigma | D8418-250ML |

### Protocol

A buffered solution of Furin reacts with the substrate sold under the brand name Rh110 Furin substrate®, to form the fluorophore Rh110 (rhodamine 110). Slight stirring for 30 seconds is carried out then the whole is incubated at room temperature for 60 minutes.

The product of the invention at three different concentrations (E1), the reference product (T+) and the control (T−) are contacted with a solution of Furin at the same time as the enzyme substrate. The activity of Furin in the presence/absence of the product of the invention or of the reference product is then evaluated.

The fluorescence intensities are collected using an excitation filter allowing wavelengths from 473 nm to 497 nm to pass (Exc485-12 filter) and an emission filter allowing wavelengths from 505 to 565 nm to pass (Em535-30 filter).

The modulation of this activity is expressed as a percentage of inhibition or activation of the activity of Furin in the absence of active ingredient, that is to say only in the presence of the substrate of the enzyme.

### Results

At the end of the incubation period, the activity of Furin with and without product (invention or reference) was evaluated by measuring the fluorescence intensities (expressed in RFU: relative fluorescence unit). For each concentration tested, the modulation of furin activity by the product under test is calculated according to the following formula.

Modulation percentage of Furin=100×[(OD−OD Furin only)/OD Furin only] [Math. 1]

If the result is negative, the percentage is expressed as enzyme inhibition, if the result is positive, the percentage is expressed as enzyme activation. The results are shown in Table 2.

TABLE 2
Average results obtained on the reference product
(T+), the control (T−) and the product of
the invention (E1) at three different concentrations.

| | | Average OD | | |
|---|---|---|---|---|
| | Average | Sample − OD | % enzyme | % inhibition of |
| Samples | OD (nm) | Blank (nm) | activity | enzyme activity |
| Blank | 44.3 | 0.0 | 0.00 | 100.00 |
| T⁺ | 540.3 | 496.0 | 100.00 | 0.00 |
| T⁻ | 149.0 | 104.7 | 21.10 | 78.90 |
| E1 at 5% | 45.0 | 4.7 | 0.94 | 99.06 |
| E1 at 2% | 49.3 | 10.3 | 2.08 | 97.92 |
| E1 at 0.5% | 121.0 | 79.3 | 15.99 | 84.01 |

At the concentrations tested of 5%, 2% and 0.5%, an inhibiting activity of Furin is observed.

### EXAMPLE 3: INHIBITION OF THE ACTIVITY OF THE ENZYME ACE2 BY THE PRODUCT OF THE INVENTION

The purpose of this study is to evaluate the modulation of the activity of the angiotensin-converting enzyme 2 (ACE2) by the product of the invention in an acellular In Vitro model using the analysis kit sold under the brand ACE2 Inhibitor screening assay kit® designed to measure the activity of the ACE2 exopeptidase.

### Material

The product of the invention was tested in triplicate at a concentration of 5%.

The reagents used are listed in Table 3 below.

TABLE 3
Used reagents

| REAGENT | SUPPLIER | REFERENCE |
|---|---|---|
| ACE2 Inhibitor screening assay kit | BPS Bioscience | 79923 |
| ACE2Enzyme Solution | BPS Bioscience | 11003 |
| ACE2 Fluorogenic substrate | BPS Bioscience | |
| ACE2 Assay Buffer | BPS Bioscience | |
| 96-well black microplate (Nunc U96 | BPS Bioscience | 79685 |
| PP0.5 ml) | | |
| DX600 | Cayman | 22186 |

### Method

An ACE2 solution is contacted with the DX600 inhibitor buffer or with the product of the invention (E1 5%) then the substrate is incorporated. The microplate is incubated at room temperature for 60 minutes. The ACE2 solution reacts with the specific substrate sold under the trademark ACE2 Fluorogenic substrate®, to form a fluorogenic compound. The fluorescence intensities are collected using an excitation filter allowing wavelengths of 544 nm to pass (Exc544 filter) and an emission filter allowing wavelengths of 580 to 600 nm to pass (Em590-10 filter). The activity of ACE2 is thus evaluated.

### Results

At the end of the incubation period, the activity of ACE2 with and without product (invention E1 5%, or reference T+ or T−) was evaluated by measuring the fluorescence intensities (expressed in RFU: relative fluorescence unit).

For each tested concentration, the modulation of the activity of ACE2 by the product of the invention or by the reference is calculated according to the following formula.

Modulation percentage of ACE2=100×[(OD−OD ACE2 only)/OD ACE2 only] [Math. 2]

The results are shown in Table 4 below.

TABLE 4
Average results obtained on the reference product (T+), the
control (T−) and the product of the invention (E1).

| | Average | Average | | |
|---|---|---|---|---|
| | fluorescence | fluorescence | | % inhibition |
| | intensities | intensity Sample − | % enzyme | of enzyme |
| Samples | (RFU) | OD Blank (RFU) | activity | activity |
| Blank | 89576.0 | 0.0 | 0.00 | — |
| T⁺ | 101450.3 | 11874.3 | 100.00 | 0.00 |
| T⁻ | 93017.7 | 3441.7 | 28.98 | 71.02 |
| E1 to 5% | 89113.7 | 1900.0 | 16.00 | 84.00 |

At the tested concentration of 5%, an inhibiting activity of ACE2 is observed.

### EXAMPLE 4: DEMONSTRATION OF THE STIMULATION OF BLOOD MICROCIRCULATION AND THE SWELLING OF CELLS BY THE PRODUCT OF THE INVENTION

### Method

The stimulation of blood microstimulation and the swelling of cells exerted by the product of the invention were evaluated with a VivaScope® 1500 brand apparatus (Reflectance Confocal Microscope) for each subject:
before the application of the product then
5 minutes and 120 minutes after a single and same application of the product of the invention.

The number of blood cells in a moving capillary during 10 s has been determined to give information about the microcirculation.

In terms of cell swelling, the average cell size was measured using the apparatus software (average over 10 cells).

### Results

The microcirculation was increased by 28%, 5 minutes after application of the product. The impact on the microcirculation has a long-term effect up to 2 hours (+10%) after application of the product.

The increase in microcirculation from the blood capillaries reflects a significant moisturizing effect on the whole of the tissue which allows to drive out the mediators of inflammation and edema and leads to a repairing effect on the nasal mucosa.

1. A product for use thereof in the treatment or prevention of the respiratory symptoms of COVID-19 disease in humans, said product comprising a hydrogel based on water, glycerol and a carbomer, and said product being in a form adapted for topical application to the respiratory epithelium.

2. The product for use thereof according to claim 1, characterized in that the hydrogel consists of water, glycerol and carbomer.

3. The product for use thereof according to claim 1, characterized in that the hydrogel represents from 10% to 30% by mass of the mass of the product.

4. The product for use thereof according to claim 1, characterized in that the carbomer comprises from 55% to 70% of carboxylic acid groups.

5. The product for use thereof according to claim 1, characterized in that the mass ratio between the carbomer and the glycerin is comprised between 1/100 and 5/100.

6. The product for use thereof according to claim 1, characterized in that the carbomer is a homopolymer of acrylic acid cross-linked with an allyl ether of pentaerythritol.

7. The product for use thereof according to claim 1, characterized in that the product is in a form adapted for topical application to the epithelium of the nasal mucosa.

8. The product for use thereof according to claim 1, characterized in that the hydrogel is capable of being obtained by suspending the carbomer in a mixture essentially consisting of water and glycerol, preferably a mixture consisting of water and glycerol.

9. A method for manufacturing the product for use thereof according to claim 1 comprising a step of preparing the hydrogel by suspending the carbomer in a mixture essentially consisting of water and glycerol.
